(19) United States
(12) Patent Application Publication
(10) Pub. No.: US 2025/0222055 A1
(43) Pub. Date: Jul. 10, 2025

(54) COMPOSITION FOR PREVENTING AND TREATING STROKE COMPRISING EXTRACT OF JUGLANS MANDSHURICA FRUIT

(71) Applicant: NEUROLMED CO., LTD, Anseong-si Gyeonggi-do (KR)

(72) Inventors: Kang Hoon JE, Anseong-si (KR); Sun Young YOON, Anseong-si (KR)

(73) Assignee: NEUROLMED CO., LTD, Anseong-si Gyeonggi-do (KR)

(21) Appl. No.: 18/877,747

(22) PCT Filed: Aug. 26, 2021
(86) PCT No.: PCT/KR2021/011456

§ 371 Date: Dec. 20, 2024

(30) Foreign Application Priority Data
Aug. 26, 2020 (KR) 10-2020-0107782
Aug. 26, 2021 (KR) 10-2021-0113156

(51) Int. Cl. A61K 36/52; A61K 31/20; A61K 31/201; A61K 31/202; A61P 9/10

(52) U.S. Cl. CPC A61K 36/52; A61K 31/20; A61K 31/201; A61K 31/202; A61P 9/10; A61K 2236/331; A61K 2236/333; A61K 2236/35; A61K 2236/37

(57) ABSTRACT

The present invention relates to a pharmaceutical composition for preventing or treating stroke, comprising a pressed extract, a solvent extract or a fraction of a solvent extract of Juglans mandshurica fruit. The Juglans mandshurica fruit exhibits a protective effect on brain tissue (brain cells) against ischemic brain tissue damage induced by middle cerebral artery occlusion and reduces infarction, and thus can be usefully used for a pharmaceutical composition or health functional food for preventing, ameliorating or treating stroke.

### TECHNICAL FIELD

The present invention relates to a composition for preventing and treating stroke.

### BACKGROUND ART

Stroke, commonly known as “cerebral apoplexy”, refers to a condition in which a blood vessel supplying blood to the brain is blocked or bursts, damaging that part of the brain, causing localized neurological disorders such as loss of consciousness, hemiplegia, communication disorders, and the like due to sudden cerebral blood flow disruption.

Stroke may be broadly divided into two types. The first is cerebral infarction, ischemic stroke, or infarct stroke, which occurs when a blood vessel is blocked and a part of the brain supplied with blood by the blood vessel is damaged. The second is cerebral hemorrhage or hemorrhagic stroke, which occurs when a cerebral blood vessel bursts and blood pools in the brain, damaging that part of the brain. Ischemic stroke occurs three times more frequently than hemorrhagic stroke.

Ischemic stroke occurs when the blood supply to the brain tissue is reduced or blocked, causing the brain tissue to become ischemic, while hemorrhagic stroke occurs when a blood vessel bursts, causing bleeding into the brain tissue. Specifically, depending on the cause, there are cerebral infarction, which is caused by blockage of a cerebral blood vessel, and cerebral hemorrhage, which is caused by rupture of a cerebral blood vessel. Cerebral hemorrhage is further divided into hypertensive intracerebral hemorrhage and subarachnoid hemorrhage (SAH) caused by rupture of a cerebral aneurysm due to the deformation of a cerebral blood vessel into a balloon shape.

Also, cerebral infarction is divided into cerebral thrombosis (a thrombus is formed in a cerebral blood vessel and blocks the blood vessel) and cerebral embolism (a thrombus fragment in a cerebral blood vessel or a blood clot caused by heart abnormality flows into the blood vessel in the head and blocks the blood vessel). The other examples thereof include small cerebral infarction (blockage of very small cerebral blood vessels), vasculitis due to encephalitis, stroke due to congenital vascular malformation, and the like.

### DISCLOSURE

### Technical Problem

The mortality rate due to stroke is increasing every year, not only domestically but also worldwide, and today, much interest focused on the development of drugs for treating stroke. The present invention is intended to provide a composition for preventing or treating stroke.

### Technical Solution

The present invention provides a pharmaceutical composition for preventing or treating stroke, including a compressed extract, a solvent extract, or a fraction of the solvent extract of Juglans mandshurica fruit.

In addition, the present invention provides a food composition for preventing or ameliorating stroke, including a compressed extract, a solvent extract, or a fraction of the solvent extract of Juglans mandshurica fruit.

In addition, the present invention provides a method treating stroke, including. of preventing or administering a compressed extract, a solvent extract, or a fraction of the solvent extract of Juglans mandshurica fruit to a subject.

In addition, the present invention provides the use of a compressed extract, a solvent extract, or a fraction of the solvent extract of Juglans mandshurica fruit for the manufacture of a medicament for the prevention or treatment of stroke.

### Advantageous Effects

A Juglans mandshurica fruit extract according to the present invention has a brain neuron protective effect and can be useful in the pharmaceutical and functional health food fields for treating, ameliorating, or preventing cerebral stroke.

Also, when the Juglans mandshurica fruit extract according to the present invention is orally administered to a middle cerebral artery occlusion animal model, infarct damage in brain slice tissue is confirmed to be minimal, and thus, the Juglans mandshurica fruit extract is effective at improving motor ability and cognitive ability or enhancing memory in addition to the brain cell protective effect.

### DESCRIPTION OF DRAWINGS

FIG. 1 shows images the cerebral infarction volume in the brain tissue of groups orally administered oil extracts of Juglans mandshurica fruit, walnut (Juglans sinensis), camellia (C. japonica L.), perilla (Perilla frutescens), and sunflower (Helianthus annuus) seeds;

FIG. 2 is a quantitative graph showing results of measurement of the cerebral infarction volume in the groups orally administered the oil extracts of Juglans mandshurica fruit, walnut (Juglans sinensis), camellia (C. japonica L.), perilla (Perilla frutescens), and sunflower (Helianthus annuus) seeds;

FIG. 3 shows gas chromatography spectra of a compressed extract of Juglans mandshurica fruit according to the present invention;

FIG. 4 shows images comparing the cerebral infarction volume in the brain tissue of the group orally administered the compressed extract of Juglans mandshurica fruit;

FIG. 5 is a quantitative graph showing results of measurement of the cerebral infarction volume in the group orally administered the compressed extract of Juglans mandshurica fruit; and

FIG. 6 is a quantitative graph showing a change in infarct volume of the mouse cerebral infarction tissue slices 1-5 of each group shown in FIG. 4.

### BEST MODE

Hereinafter, a detailed description will be given of the present invention.

Meanwhile, each description and embodiment disclosed in the present invention may also be applied to other descriptions and embodiments thereof. Briefly, all combinations of various elements disclosed in the present invention fall within the scope of the present invention. Also, the scope of the present invention should not be considered as limited by the specific description given below.

Expressions such as “including” as used herein should be understood as open-ended terms implying the possibility of including other embodiments, unless specifically stated otherwise in the phrase or sentence in which the expression is included.

The terms or words used in the description and claims of the present invention should not be construed as limited to usual or dictionary meanings thereof, but should be interpreted as having meanings and concepts that conform to the technical idea of the present invention, based on the principle that the inventor may appropriately define the concept of the term in order to explain his or her own invention in the best manner.

An aspect of the present invention provides a pharmaceutical composition for preventing treating stroke, including a compressed extract, a solvent extract, or a fraction of the solvent extract of Juglans mandshurica fruit.

Juglans mandshurica is a tree belonging to the Juglandaceae family. It is a deciduous broad-leaved tree with pinnate compound leaves, 7 to 17 small leaves, each about 7 to 28 cm long, and the fruit thereof is an egg-shaped drupe. The fruit ripens in September to October and may be used for medicinal or edible purposes.

As used herein, the term “extract” includes a liquid extract itself and an extract of any formulation that may be formed using the liquid extract, such as a liquid extract obtained by extraction treatment, a diluted or concentrated liquid extract, a dried product obtained by drying the liquid extract, a crude-purified or purified product of the liquid extract, or a mixture thereof.

In the present invention, drying of the extract may be carried out by a known method within a range in which useful components from the collected plant are not destroyed, and may be carried out, for example, by a method of natural drying in a shaded place. Also, crushing or grinding may be performed to powder the plant to the extent that the useful components of the plant may be sufficiently extracted during the subsequent extraction process. The drying and crushing or grinding processes may be performed in reverse order or repeatedly as needed.

In the extraction of the present invention, the extraction method is not particularly limited, and extraction may be performed according to a method commonly used in the relevant technical field. Non-limiting examples of the extraction method include solvent extraction, ultrasonic extraction, filtration, reflux extraction, compression, and the like, which may be performed alone or in combination of two or more thereof.

“Compression” is a method of squeezing out components such as oil by compressing a material. This method includes separating oil by compressing Juglans mandshurica fruit, which allows for obtaining fresh, high-quality oil with less heat-related deterioration and damage.

The compressed extract of Juglans mandshurica fruit may be obtained by separating oil from Juglans mandshurica fruit by compression at a temperature of 15 to 120° C. under a pressure of 10 to 4,000 kg/cm², and specifically, the oil may be separated from Juglans mandshurica fruit by compression at a temperature of 20 to 100° C. under a pressure of 500 to 3,000 kg/cm².

In the present invention, Juglans mandshurica fruit may be prepared into a solvent extract using a solvent, and the type of extraction solvent used for extraction is not particularly limited, and any solvent known in the art may be used. In the present invention, the extract may be obtained by extraction with water, a C₁-C₄ lower alcohol, hexane, or a mixed solvent thereof. In addition, non-limiting examples of the extraction solvent include water; C₁-C₄ lower alcohols such as methanol, ethanol, propyl alcohol, butyl alcohol, etc.; polyhydric alcohols such as glycerin, butylene glycol, propylene glycol, etc.; hydrocarbon-based solvents such as methyl acetate, ethyl acetate, acetone, benzene, hexane, diethyl ether, dichloromethane, etc.; or mixtures thereof. Specifically, water, lower alcohols, 1,3-butylene glycol, ethyl acetate, and hexane may be used alone or in combination of two or more thereof.

The term “fraction” used herein refers to the result obtained by performing fractionation to separate a specific component or a specific component group from a mixture including various components.

The fractionation method for obtaining the fraction in the present invention is not particularly limited, and may be performed according to a method commonly used in the relevant technical field. A non-limiting example of the fractionation method may include a method of treating a solvent extract of Juglans mandshurica fruit with a predetermined solvent to obtain a fraction from the extract. The type of solvent used to obtain the fraction in the present invention is not particularly limited, and any solvent known in the art may be used. Non-limiting examples of the fractionation solvent include water, C₁-C₄ alcohols, hexane, ethyl acetate, chloroform, dichloromethane, or a mixed solvent thereof, and fractions of the solvent extract of Juglans mandshurica fruit may be prepared by fractionation with these solvents.

As used herein, the term “stroke” refers to a local neurological deficit symptom suddenly induced by cerebral blood flow abnormality. The stroke may include cerebral infarction, which occurs when a cerebral blood vessel is blocked by a thrombus, etc., causing brain cell death, or cerebral hemorrhage, which occurs when a cerebral blood vessel ruptures. Specific symptoms of stroke include sudden headache and vomiting, hemiplegia or paralysis of part of the body, numbness and loss of sensation in part of the body, communication disorders (aphasia or pronunciation disorders), facial nerve disorders, ataxia, and the like. In the present invention, stroke may mean cerebral apoplexy.

The stroke may be an ischemic stroke or a hemorrhagic stroke, and particularly, may be an ischemic stroke.

The pharmaceutical composition of the present invention may serve to promote recovery of motor function damaged due to stroke or to reduce infarction size.

The term “prevention” used herein refers to any action of inhibiting or delaying the onset of stroke by administering a composition including the Juglans mandshurica fruit extract according to the present invention. The term “treatment” used herein refers to any action of alleviating or beneficially changing the symptoms of the disease by administering a composition including the Juglans mandshurica fruit extract according to the present invention.

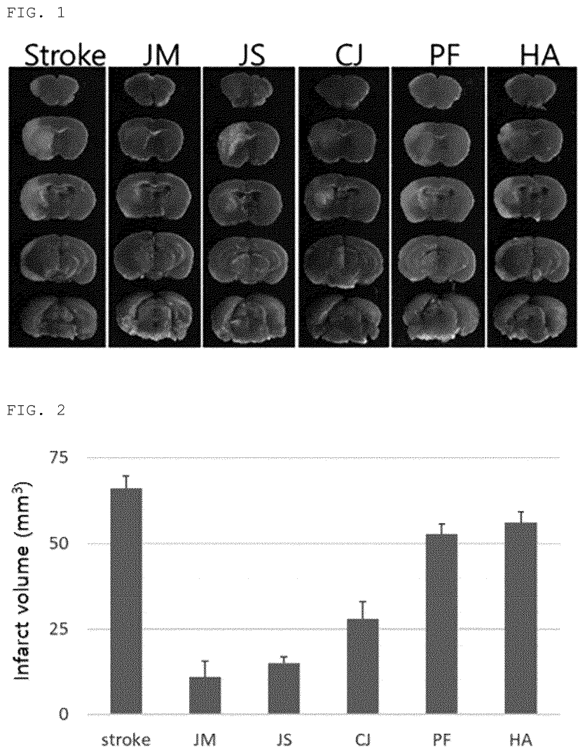

In a specific embodiment of the present invention, the infarction size was confirmed to decrease when a stroke animal model was treated with the compressed extract or hexane extract of Juglans mandshurica fruit according to the present invention (FIGS. 1, 2, and 4-6).

The Juglans mandshurica fruit extract, which is effective at preventing or treating stroke as described above, includes fatty acids, and the fatty acids may include oleic acid, linoleic acid, and linolenic acid in a weight ratio of 1-2.5:4-8:1, and more specifically, oleic acid, linoleic acid, and linolenic acid in a weight ratio of 1.25-1.35:4.5-6.8:1.

In addition, the Juglans mandshurica fruit extract includes other fatty acids in addition to oleic acid, linoleic acid, and linolenic acid, and may include 1.98 to 2.98 wt % of palmitic acid, 0.53 to 0.79 wt % of stearic acid, 9.08 to 13.62 wt % of oleic acid, 44.83 to 67.25 wt % of linoleic acid, and 6.93 to 10.39 wt % of linolenic acid based on the total weight of fatty acids.

In an embodiment of the present invention, among the fatty acids, the Juglans mandshurica fruit extract had a ratio of oleic acid (C18:1, ω9) to linoleic acid (C18:2, ω6) to linolenic acid (C18:3, ω3) of about 1.07:4.92:1, walnut had a ratio of about 2.36:6.63:1, camellia had a ratio of about 851:29:1, perilla had a ratio of about 0.29:0.18:1, and sunflower seeds had a ratio of about 142.83:601.33:1.

The ratio of oleic acid to linoleic acid to linolenic acid among the fatty acids contained in the Juglans mandshurica fruit and walnut oils was about 1-2.5:4-8:1, but camellia had a relatively high proportion of oleic acid, sunflower seeds had a significantly high proportion of linoleic acid, and perilla had significantly low proportions of oleic acid and linoleic acid.

Also, based on results of measurement of the cerebral infarction treatment effect using these oil extracts, the cerebral infarction volume reduction effect was vastly superior in the oil extracts of Juglans mandshurica fruit and walnut including oleic acid, linoleic acid, and linolenic acid in a weight ratio of 1-2.5:4-8:1. Thereby, when including oleic acid, linoleic acid, and linolenic acid as the main fatty acids in a weight ratio of 1-2.5:4-8:1, the oil extract has the effect of preventing or treating stroke. More specifically, the extract in which the oleic acid, linoleic acid, and linolenic acid are included in a weight ratio of 1.25-1.35:4.5-6.8:1 has high activity of preventing or treating stroke.

Also, the composition may a further include walnut extract, thereby enhancing the activity of preventing or treating stroke.

The walnut extract may be a compressed extract, a solvent extract, or a fraction of the solvent extract of walnut (Juglans sinensis), and specifically may be an oil extract of walnut. A description of the compressed extract, the solvent extract, and the fraction of the solvent extract of walnut fruit is the same as the above description of the compressed extract, the solvent extract, and the fraction of the solvent extract of Juglans mandshurica.

Among the fatty acids included in walnut, the ratio of oleic acid to linoleic acid to linolenic acid may be 1-5:4-12:1, and specifically, when the weight ratio thereof falls in the range of 1.25-4.5:4.5-10:1, the activity of preventing or treating stroke is excellent.

The pharmaceutical composition according to the present invention may contain the Juglans mandshurica fruit extract of the present invention alone or may further contain at least one pharmaceutically acceptable carrier, excipient, or diluent.

The composition for preventing treating or stroke according to the present invention may be prepared into a pharmaceutical formulation using methods well known in the art so as to provide rapid, sustained, or delayed release of the active ingredient after administration to a mammal. In the preparation of a formulation, the active ingredient may be mixed or diluted with a carrier, or may be enclosed in a carrier in the form of a container. Therefore, the composition for preventing or treating stroke according to the present invention may be prepared and used in the form of oral formulations such as powders, granules, tablets, capsules, suspensions, emulsions, syrups, aerosols, etc., external preparations, suppositories, and sterile injectable solutions by typical methods, and may further include an appropriate carrier, excipient, and diluent typically used in the preparation of compositions.

For example, examples of the carrier that may be included in the composition according to the present invention include, but are not limited to, lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methyl hydroxybenzoate, propyl hydroxybenzoate, talc, magnesium stearate, and mineral oil. The formulations are prepared using typical diluents or excipients such as fillers, extenders, binders, wetting agents, disintegrants, surfactants, etc.

Solid preparations for oral administration include tablets, pills, powders, granules, and capsules, and these solid preparations are prepared by mixing the active ingredient of the present invention with at least one excipient, such as starch, calcium carbonate, sucrose, lactose, and gelatin. In addition to simple excipients, lubricants may also be used.

Liquid preparations for oral administration include suspensions, solutions, emulsions, and syrups. In addition to the commonly used simple diluents such as water and liquid paraffin, various excipients such as wetting agents, sweeteners, fragrances, and preservatives may be included.

Formulations for parenteral administration include sterile aqueous solutions, non-aqueous solutions, suspensions, emulsions, lyophilized preparations, and suppositories. Non-aqueous solutions and suspensions may include propylene glycol, polyethylene glycol, vegetable oils such as olive oil, injectable esters such as ethyl oleate, and the like. As a base for suppositories, Witepsol, Macrogol, Tween 61, cacao butter, laurin oil, glycerogelatin, etc. may be used.

The pharmaceutical composition according to the present invention may be administered as an individual therapeutic agent or in combination with another therapeutic agent, and may be administered sequentially or simultaneously with a conventional therapeutic agent. The pharmaceutical composition may be administered single or multiple times. Considering all of the factors, it is important to administer an amount capable of achieving the maximum effect with the minimum amount without causing side effects, which may be easily determined by a person skilled in the art.

As used herein, the term “administration” means introducing the pharmaceutical composition according to the present invention into a patient by any appropriate method, and various routes of administration of the composition according to the present invention, such as oral or parenteral administration, may be employed so long as it may reach the target tissue.

The daily dosage of the pharmaceutical composition according to the present invention is usually in the range of 0.001 to 150 mg/kg body weight, and may be administered once or in several divided doses. However, since the actual dosage of the pharmaceutical composition according to the present invention is determined depending on various related factors such as the route of administration, the patient's age, gender, body weight, and the patient's severity, the dosage should not be construed as limiting the scope of the present invention in any way.

Another aspect of the present invention provides a food composition for preventing or ameliorating stroke, including a compressed extract, a solvent extract, or a fraction of the solvent extract of Juglans mandshurica fruit.

The terms “Juglans mandshurica”, “compressed extract”, “solvent extract”, “fraction”, “stroke”, and “prevention” used herein are as described above.

The Juglans mandshurica fruit extract according to the present invention serves to reduce the cerebral infarction volume in a middle cerebral artery occlusion model, and thus has a protective effect on the brain, such as brain cells or brain tissue.

The term “amelioration” used herein refers to any action of alleviating or benefiting the symptoms of a subject suspected of or suffering from stroke using the composition including the Juglans mandshurica fruit extract according to the present invention.

The food composition for preventing or ameliorating stroke according to the present invention is provided in forms such as pills, powders, granules, infusions, tablets, capsules, or liquids, and examples of foods to which the composition of the present invention may be added include various foods, such as beverages, gum, tea, vitamin complexes, health supplements, and the like.

There is no particular limitation on other ingredients in addition to the composition or the Juglans mandshurica fruit extract, the active ingredient thereof, as an essential ingredient that may be included in the food composition for preventing or ameliorating stroke according to the present invention, and various herbal extracts, food additives, or natural carbohydrates may be contained as additional ingredients like typical foods.

The food additives include food additives commonly used in the art, such as flavoring agents, seasonings, coloring agents, fillers, stabilizers, and the like.

Examples of the natural carbohydrates include monosaccharides such as glucose, fructose, etc.; disaccharides such as maltose, sucrose, etc.; and polysaccharides such as dextrin, cyclodextrin, etc., and sugar alcohols such as xylitol, sorbitol, erythritol, etc. In addition thereto, natural flavoring agents (thaumatin, etc.), stevia extracts (rebaudioside A, glycyrrhizin, etc.), and synthetic flavoring agents (saccharin, aspartame, etc.) may be used.

The health supplements include functional health foods and health foods.

The functional health food is the same term as food for special health use (FoSHU), and refers to a food with high medical and healthcare effects processed to efficiently exhibit bioregulatory functions in addition to providing nutrition. Here, “functional (functionality)” means obtaining an effect useful for health purposes, such as regulating nutrients or physiological actions, for the structure and function of the human body. The food of the present invention may be prepared by a method commonly used in the art, and during the preparation process, raw materials and ingredients commonly used in the art may be added. Also, the formulation of the food may be prepared without limitation, so long as it is a formulation recognized as food. The food composition of the present invention may be prepared in various formulation forms, and has the advantage of not having side effects that may occur when taking drugs for a long period of time because it uses food as a raw material, unlike general drugs. Also, since it is highly portable, the food of the present invention may be consumed as a supplement to enhance the effect of preventing or ameliorating stroke.

Still another aspect of the present invention provides a method of preventing or treating stroke, including administering a compressed extract, a solvent extract, or a fraction of the solvent extract of Juglans mandshurica fruit to a subject.

The prevention or treatment method of the present invention may specifically include administering a pharmaceutically effective amount of a compressed extract, a solvent extract, or a composition including the solvent extract of Juglans mandshurica fruit to a subject who has suffered from or is at risk of suffering from stroke.

The terms “Juglans mandshurica”, “compressed extract”, “solvent extract”, “fraction”, “stroke”, “prevention”, and “treatment” used herein are as described above.

The term “subject” used herein refers to any animal, including humans, which has suffered from or is likely to suffer from stroke. The animal may be a mammal such as a cow, horse, sheep, pig, goat, camel, antelope, dog, cat, etc. in need of treatment for symptoms similar thereto as well as a human, but a subject in which stroke is prevented or treated by the compressed extract, the solvent extract, or the fraction of the solvent extract of Juglans mandshurica fruit is included without limitation.

Yet another aspect of the present invention provides the use of a compressed extract, a solvent extract, or a fraction of the solvent extract of Juglans mandshurica fruit for the manufacture of a medicament for the prevention or treatment of stroke.

The terms “Juglans mandshurica”, “compressed extract”, “solvent extract”, “fraction”, “stroke”, “prevention”, and “treatment” used herein are as described above.

Specifically, the present invention provides the use of a composition including a compressed extract, a solvent extract, or a fraction of the solvent extract of Juglans mandshurica fruit for the manufacture of a medicament for the prevention or treatment of stroke.

### MODE FOR THE INVENTION

Below, examples of the present invention will be described in detail with reference to the attached drawings so that the present invention may be easily implemented by a person having ordinary skill in the art to which the present invention pertains. However, the present invention may be embodied in many different forms and is not limited to the examples described herein.

### Experimental Example 1. Preparation of Various Oil Extracts and Analysis of Fatty Acid Components

### 1-1: Preparation of Oil Extract

To analyze the fatty acid composition of Juglans mandshurica fruit, walnut (Juglans sinensis), camellia (C. japonica L.), perilla (Perilla frutescens), and sunflower (Helianthus annuus) seeds, oil extracts of Juglans mandshurica fruit, walnut (Juglans sinensis), camellia (C. japonica L.), perilla (Perilla frutescens), and sunflower (Helianthus annuus) seeds were prepared or purchased. Juglans mandshurica fruit was compressed at 45-92° C. under a pressure of 2,000 kg/cm² to provide an oil extract. Walnut (Juglans sinensis) and sunflower (Helianthus annuus) seed oil extracts were prepared by adding chloroform and methanol in a volume ratio of 2:1 (v/v). Camellia (C. japonica L.) oil was also prepared by compression, and commercially available compressed perilla (Perilla frutescens) oil was used.

### 1-2. Analysis of Fatty Acid Components

The oil extracts of Juglans mandshurica fruit, walnut (Juglans sinensis), camellia (C. japonica L.), perilla (Perilla frutescens), and sunflower (Helianthus annuus) seeds prepared in Experimental Example 1-1 were lyophilized and then finely powdered. A certain amount of sample was placed in a 4 ml vial with a Teflon cap, and 0.5 ml of pentadecanoic acid (1 mg/ml hexane) was also added as an internal standard. Thereafter, 2 ml of a methylation mixture [MeOH:benzene:DMP (2,2-dimethoxy-propane):H₂SO₄=39:20:5:2] and 1 ml of heptane were added and shaken, followed by extraction at 80° C. for 2 hours. After extraction, a certain amount of the supernatant was separated from the two layers formed by cooling to room temperature and then analyzed using gas chromatography. Fatty acid analysis of these oil extracts was performed using an Agilent Technologies 7890A gas chromatography (GC) system (Agilent Technologies, Santa Clara, CA, USA), a DB-23 capillary column (Agilent Technologies, 60 m×0.25 mm i.d., 0.25-μm film thickness), and nitrogen (2.7 ml/min) as the carrier gas.

The inlet temperature was 250° C., the detector temperature was 280° C., the inlet sample injection ratio was set to 10:1, and respective speeds of hydrogen and air for flame ionization in the detector were set to 35 ml/min and 350 ml/min. The oven temperature program was initially set at 50° C. for 1 minute, then increased by 25° C. per minute to 130° C., 8° C. per minute to 170° C., 1.5° C. per minute to 215° C., and 5° C. per minute to 250° C., and maintained for 5 minutes.

The analyzed results were confirmed using respective retention times using 37 fatty acid standards (Supelco 37 FAME Mix, Supelco, USA).

Table 1 below shows the gas chromatography spectra of the oil extracts of Juglans mandshurica fruit, walnut (Juglans sinensis), camellia (C. japonica L.), perilla (Perilla frutescens), and sunflower (Helianthus annuus) seeds. As shown in Table 1, the retention time (Rt) for each fatty acid was measured and the fatty acid composition of these oil extracts was analyzed using gas chromatography. The results thereof are shown in Table 1 below.

TABLE 1

| | | Comparative | Comparative | Comparative | Comparative |
|---|---|---|---|---|---|
| | Example 1 | Example 1 | Example 2 | Example 3 | Example 4 |
| Total fatty | Juglans | Walnut oil | Camellia oil | Perilla oil | Sunflower |
| acid | mandshurica | | | | seed oil |
| composition | fruit oil | | | | |
| (wt %) | | | | | |
| C14:0 | N.D. | N.D. | N.D. | N.D. | 0.08 |
| Palmitic | 2.71 | 6.33 | 8.90 | 5.66 | 6.14 |
| acid | | | | | |
| (C16:0) | | | | | |
| C17:0 | N.D. | 0.10 | N.D. | N.D. | 0.06 |
| Stearic | 0.81 | 1.90 | 2.40 | 1.90 | 3.83 |
| acid | | | | | |
| (C18:0) | | | | | |
| Arachidonic | N.D. | 0.07 | N.D. | 0.13 | 0.26 |
| acid | | | | | |
| (C20:0) | | | | | |
| Palmitoleic | N.D. | 0.05 | N.D. | N.D. | 0.06 |
| acid | | | | | |
| (C16:1ω7) | | | | | |
| Oleic acid | 14.79 | 21.63 | 85.10 | 18.06 | 17.14 |
| (C18:1ω9) | | | | | |
| Linoleic | 67.68 | 60.62 | 2.90 | 11.32 | 72.16 |
| acid | | | | | |
| (C18:2ω6) | | | | | |
| Linolenic | 13.76 | 9.15 | 0.10 | 62.29 | 0.12 |
| acid | | | | | |
| (C18:3ω3) | | | | | |
| C20:1 | 0.24 | 0.16 | 0.30 | 0.14 | 0.14 |

(N.D. stands for “not detected” and indicates that the corresponding ingredient was not detected.) These oil extracts contained the major fatty acids in common, such as palmitic acid, stearic acid, oleic acid, linoleic acid, and linolenic acid, and compositions thereof are shown in Table 2 below.

TABLE 2

| Total fatty | | | | | |
|---|---|---|---|---|---|
| acid | Juglans | | | | |
| composition | mandshurica | Walnut | Camellia | Perilla | Sunflower |
| (wt %) | fruit oil | oil | oil | oil | seed oil |
| Palmitic acid | 2.71 | 6.33 | 8.90 | 5.66 | 6.14 |
| (C16:0) | | | | | |
| Stearic acid | 0.81 | 1.90 | 2.40 | 1.90 | 3.83 |
| (C18:0) | | | | | |
| Oleic acid | 14.79 | 21.63 | 85.10 | 18.06 | 17.14 |
| (C18:1ω9) | | | | | |
| Linoleic acid | 67.68 | 60.62 | 2.90 | 11.32 | 72.16 |
| (C18:2ω6) | | | | | |
| Linolenic acid | 13.76 | 9.15 | 0.10 | 62.29 | 0.12 |
| (C18:3ω3) | | | | | |

In addition, these oil extracts had high amounts of oleic acid, linoleic acid, and linolenic acid among five fatty acids including palmitic acid, stearic acid, oleic acid, linoleic acid, and linolenic acid, and the ratios of oleic acid to linoleic acid to linolenic acid are shown in Table 3 below. The fruit of Juglans mandshurica contained fatty acids in a ratio of oleic acid (C18:1, ω9) to linoleic acid (C18:2, ω6) to linolenic acid (C18:3, ω3) of about 1.07:4.92:1, walnut contained fatty acids in a ratio of about 2.36:6.63:1, camellia contained fatty acids in a ratio of about 851:29:1, perilla contained fatty acids in a ratio of about 0.29:0.18:1, and sunflower seeds contained fatty acids in a ratio of about 142.83:601.33:1. Overall, the Juglans mandshurica fruit and walnut oil extracts had a ratio of oleic acid to linoleic acid to linolenic acid of about 1-2.5:4.5-6.8:1.

TABLE 3

| Omega fatty | | | | | |
|---|---|---|---|---|---|
| acid | Juglans | | | | |
| composition | mandshurica | Walnut | Camellia | Perilla | Sunflower |
| (wt %) | fruit oil | oil | oil | oil | seed oil |
| Oleic acid | 14.79 | 21.63 | 85.10 | 18.06 | 17.14 |
| (C18:1ω9) | | | | | |
| Linoleic acid | 67.68 | 60.62 | 2.90 | 11.32 | 72.16 |
| (C18:2ω6) | | | | | |
| Linolenic acid | 13.76 | 9.15 | 0.10 | 62.29 | 0.12 |
| (C18:3ω3) | | | | | |
| Omega fatty | | | | | |
| acid | | | | | |
| weight ratio | | | | | |
| Oleic acid | 1.07 | 2.36 | 851.00 | 0.29 | 142.83 |
| (C18:1ω9) | | | | | |
| Linoleic acid | 4.92 | 6.63 | 29.00 | 0.18 | 601.33 |
| (C18:2ω6) | | | | | |
| Linolenic acid | 1.00 | 1.00 | 1.00 | 1.00 | 1.00 |
| (C18:3ω3) | | | | | |

### Experimental Example 2. Comparison of Effects of Various Oil Extracts on Treating Stroke

To confirm the stroke treatment effects of the oil extracts of Juglans mandshurica fruit, walnut (Juglans sinensis), camellia (C. japonica L.), perilla (Perilla frutescens), and sunflower (Helianthus annuus) seeds prepared in Experimental Example 1, a middle cerebral artery occlusion (MCAO)-induced cerebral ischemia experimental model was created and a reduction in the infarct volume was confirmed.

### 2-1. Preparation of Experimental Animals

11-week-old C57BL/6 (DBL, Korea) male mice weighing 25 g or less were purchased as experimental animals, and after an adaptation period of about one week, animal experiments were performed. The animals were allowed to adapt to the experimental environment while being provided with sufficient feed and water.

### 2-2. Creation of Middle Cerebral Artery Occlusion (MCAO) Animal Model

To damage neurons by focal cerebral ischemia, an intraluminal suture method was performed (Zea Longa, et al., Stroke, 20:84-91, 1989). Specifically, mice (C57BL/6, male, 12-13 weeks old) were anesthetized by inhalation with 2% isoflurane (70% N₂O and 30% O₂). Thereafter, while monitoring cerebral blood flow using a laser doppler, the skin in the anterior central part of the neck was incised, the common right carotid artery (CCA) and the external carotid artery (ECA) were separated, and finally the middle cerebral artery (MCA) was occluded using a silicone-coated filament. The occlusion was maintained for 1 hour, and the filament was removed after 1 hour induce reperfusion. Thereafter, the body the mice was temperature of maintained at 37±0.5° C.

2-3. Oral Administration of Compressed Extract of Juglans mandshurica Fruit

Immediately after reperfusion, the oil extracts of Juglans mandshurica fruit, walnut (Juglans sinensis), camellia (C. japonica L.), perilla (Perilla frutescens), and sunflower (Helianthus annuus) seeds prepared in Experimental Example 1 were orally administered at a concentration of 1,000 mg/kg in a volume of 50 to 100 μl per 25 g of mouse body weight. The control group was administered the same volume of saline.

### 2-4. Identification of Infarction Area

After 24 hours of reperfusion, the mice were sacrificed by euthanasia and perfused intracardially with PBS to remove blood, and brains were removed and sectioned into 2 mm slices from the frontal pole of the brain. The slices were placed in a 2% TTC (2,3,5-triphenyltetrazolium chloride) solution, stained at room temperature for 20 minutes, fixed in a paraformaldehyde solution, and photographed with a digital camera, and the infarct volume was measured using the ImageJ program.

FIG. 1 shows images comparing the cerebral infarction volume in the brain tissue of groups orally administered the oil extracts of Juglans mandshurica fruit, walnut (Juglans sinensis), camellia (C. japonica L.), perilla (Perilla frutescens), and sunflower (Helianthus annuus) seeds.

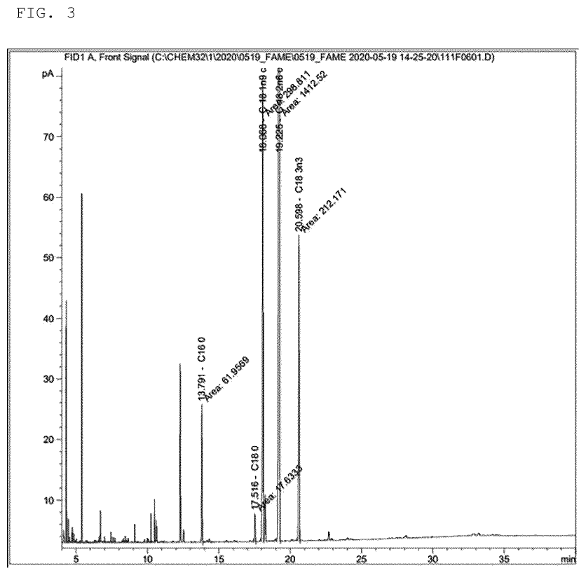

FIG. 2 is a quantitative graph showing results of measurement of the cerebral infarction volume in the groups orally administered the oil extracts of Juglans mandshurica fruit, walnut (Juglans sinensis), camellia (C. japonica L.), perilla (Perilla frutescens), and sunflower (Helianthus annuus) seeds.

The Stroke group is a group in which stroke was induced by occluding the middle cerebral artery, the JM group is a group in which stroke was induced by occluding the middle cerebral artery and then which was orally administered the oil extract of Juglans mandshurica fruit, the JS group is a group in which stroke was induced by occluding the middle cerebral artery and then which was orally administered the oil extract of walnut (Juglans sinensis), the CJ group is a group in which stroke was induced by occluding the middle cerebral artery and then which was orally administered the oil extract of camellia (C. japonica L.), the PF (perilla oil) group is a group in which stroke was induced by occluding the middle cerebral artery and then which was orally administered the compressed oil extract of perilla (Perilla frutescens), and the HA group is a group in which stroke was induced by occluding the middle cerebral artery and then which was orally administered the sunflower (Helianthus annuus) seed oil extract.

Thereby, as shown in FIG. 1, the stroke-induced group (Stroke) was found to have cerebral infarction, resulting in a white infarction area. Here, in the groups orally administered the oil extract of Juglans mandshurica fruit and the walnut oil extract, the infarct volume was significantly reduced and the intact red area was increased. Also, the camellia (C. japonica L.) oil extract also showed a reduction in cerebral infarction volume. However, the perilla oil extract and the sunflower seed oil extract had little effect on reducing the cerebral infarction volume.

Based on results fatty acid composition of analysis in Experimental Example 1, the fatty acid composition of Juglans mandshurica fruit had a ratio of oleic acid (C18:1, ω9) to linoleic acid (C18:2, ω6) to linolenic acid (C18:3, ω3) of about 1.07:4.92:1, walnut had a ratio of 2.36:6.63:1, camellia had a ratio of 851:29:1, perilla had a ratio of 0.29:0.18:1, and sunflower seeds had a ratio of 142.83:601.33:1. In the Juglans mandshurica fruit and walnut, the ratio of oleic acid (ω9) to linoleic acid (ω6) based on linolenic acid (ω3) was 1-2.5:4.5-6.8, whereas the ratio of oleic acid (ω9) to linoleic acid (ω6) in camellia was 851:29, showing a significantly higher proportion of oleic acid. In addition, sunflower seeds had a very high linoleic acid proportion as in 142.83:601.33, and perilla had very low oleic acid and linoleic acid proportions as in 0.29:0.18. Therefore, when the ratio of oleic acid to linoleic acid to linolenic acid among the fatty acids contained in the plant oil was 1-2.5:4.5-6.8:1 (weight ratio), the oil was confirmed to be effective against cerebral infarction.

### Experimental Example 3. Preparation of Compressed Extract of Juglans mandshurica Fruit and Analysis of Fatty Acid Components

3-1. Preparation of Compressed Extract of Juglans mandshurica Fruit

Juglans mandshurica fruit was prepared by purchasing commercially available Juglans mandshurica fruit. The purchased Juglans mandshurica fruit was washed thoroughly with water, sufficiently dried, and compressed under a pressure of 2,000 kg/cm² at 25° C., which is a lower temperature than in Experimental Example 1-1, obtaining a compressed extract of natural Juglans mandshurica fruit.

3-2. Component Analysis of Compressed Extract of Juglans mandshurica Fruit

Using the compressed extract of Juglans mandshurica fruit prepared in 3-1 above, fatty acid components were analyzed in the same manner as the fatty acid component analysis method used in Experimental Example 1-2.

FIG. 3 shows the gas chromatography spectra of the compressed extract of Juglans mandshurica fruit according to the present invention. As shown in FIG. 3, the retention time (Rt) for fatty each acid was determined to be palmitic acid (Rt: 13.791), stearic acid (Rt: 17.516), oleic acid (Rt: 18.668), linoleic acid (Rt: 19.225), and linolenic acid (Rt: 20.598). The results of analysis of the compressed extract of Juglans mandshurica fruit prepared in Experimental Example 3-2 using gas chromatography are shown in Table 4 below.

TABLE 4

| Fatty acid composition | Amount (wt %) |
|---|---|
| Palmitic acid C16:0 | 2.48 ± 0.01 |
| Stearic acid C18:0 | 0.66 ± 0.01 |
| Oleic acid C18:1ω9 | 11.35 ± 0.03 |
| Linoleic acid C18:2 ω6 | 56.04 ± 0.09 |
| Linolenic acid C18:3 ω3 | 8.66 ± 0.02 |

As shown in Table 4, the compressed extract of Juglans mandshurica fruit contained various fatty acids, and the ratio of oleic acid (C18:1, ω9) to linoleic acid (C18:2, ω6) to linolenic acid (C18:3, ω3) was about 1.31:6.47:1. Therefore, although there was a difference in the extraction method using hexane and the fatty acid contents in Experimental Example 1, the ratio of oleic acid to linoleic acid to linolenic acid fell within the ratio of 1-2.5:4.5-6.8:1. When the oil extract of Juglans mandshurica fruit was prepared by compression at a lower temperature than in Experimental Example 1-1, the ratio of oleic acid to linoleic acid was confirmed to increase compared to linolenic acid.

### Experimental Example 4. Analysis of Effect of Treating Stroke

A middle cerebral artery occlusion (MCAO)-induced cerebral ischemia experimental model was created in the same manner as in Experimental Example 2, and immediately after reperfusion, the compressed extract of Juglans mandshurica fruit was orally administered at a concentration of 1,000 mg/kg in a volume of 50 to 100 μl per 25 g of mouse body weight, and the effects of reducing cerebral infarction volume and protecting brain neurons were confirmed.

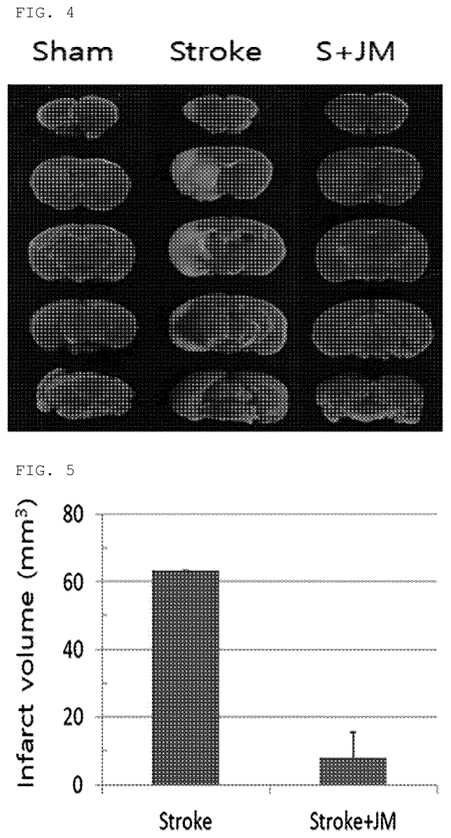

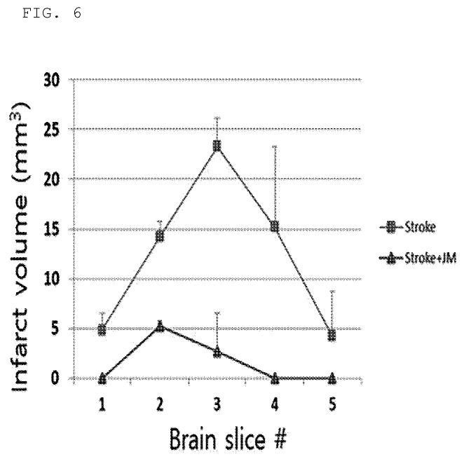

FIG. 4 shows images comparing the cerebral infarction volume in the brain tissue of the group orally administered the compressed extract of Juglans mandshurica fruit. FIG. 5 is a quantitative graph showing results of measurement of the cerebral infarction volume in the group orally administered the compressed extract of Juglans mandshurica fruit.

The Sham group is a negative control group in which the middle cerebral artery was not occluded, the Stroke group is a group in which stroke was induced by occluding the middle cerebral artery, and the S+JM group is a group in which stroke was induced by occluding the middle cerebral artery and then which was orally administered the compressed extract of Juglans mandshurica fruit.

Thereby, as confirmed in FIG. 4, the Sham group (negative control group) underwent stroke surgery, but since the middle cerebral artery was not actually occluded, cerebral infarction did not occur, so the tissue slice appeared as a red area, and the stroke-induced group (Stroke) induced cerebral infarction, so the infarction area appeared as a white area. In addition, in the group orally administered the compressed extract of Juglans mandshurica fruit after inducing cerebral infarction, cerebral infarction was recovered and reduced, as represented by the red area.

Also, as confirmed in FIG. 5, the stroke-induced group (Stroke) showed the infarct volume of 60 mm³ or more on average. In contrast, the group (S+JM) that was orally administered the compressed extract of Juglans mandshurica fruit showed the infarct volume of 10 mm³ or less, indicating that stroke was significantly reduced by administration of the compressed extract of Juglans mandshurica fruit.

FIG. 6 is a quantitative graph showing a change in the infarct volume of the mouse cerebral infarction tissue slices 1-5 of each group shown in FIG. 4.

As confirmed in the images of FIG. 4 and in FIG. 6 showing the quantitative graph thereof, tissue damage in slices 2 and 3 was significant in the stroke-induced group (Stroke). Slices 1 and 4 were found to have less tissue damage because they were relatively far from the cerebral infarction core. Also, it was confirmed for the group administered the compressed extract of Juglans mandshurica fruit (S+JM) that tissue damage was greatly reduced even in slices 2 and 3 where tissue damage was severe.

The results suggest that the Juglans mandshurica fruit extract is capable of treating, ameliorating, or preventing stroke.

### Experimental Example 5. Preparation of Compressed Extract of Walnut and Analysis of Fatty Acid Components

Compressed extracts of various types of commercially available walnut were prepared and the fatty acid components thereof were analyzed. Thereby, the compressed oil extracts of walnut contained the major fatty acids in common, such as palmitic acid, stearic acid, oleic acid, linoleic acid, and linolenic acid, but the proportions of oleic acid, linoleic acid, and linolenic acid varied depending on the type of walnut, and the ratio of fatty acids also varied in the range of 0.5-3:0.5-8:1 depending on the type of walnut.

### Experimental Example 6. Analysis of Effect of Treating Stroke

Using the compressed oil extract of walnut prepared in Experimental Example 5, a middle cerebral artery occlusion (MCAO)-induced cerebral ischemia experimental model was created in the same manner as in Experimental Example 4, and the effects of treating cerebral infarction were compared.

Thereby, when the weight ratio of oleic acid to linoleic acid to linolenic acid contained in the walnut oil fell out of 1-5:4-12:1, there was little effect on reducing cerebral infarction volume. In particular, when the weight ratio of oleic acid to linoleic acid to linolenic acid contained in the walnut oil was 1.25-4.5:4.5-10:1, the cerebral infarction volume was effectively reduced.

Although the preferred embodiments of the present invention have been described in detail above, the scope of the present invention is not limited thereto, and various modifications and improvements made by those skilled in the art using the basic concept of the present invention defined in the following claims also fall within the scope of the present invention.

1. A method of treating stroke in a subject in need thereof, comprising administering to the subject an effective amount of a pharmaceutical composition comprising a compressed extract, a solvent extract, or a fraction of the solvent extract of Juglans mandshurica fruit as an active ingredient.

2. The method according to claim 1, wherein the compressed extract is obtained by compression extraction at a temperature of 15 to 100° C. under a pressure of 10 to 4,000 kg/cm².

3. The method according to claim 1, wherein the solvent extract is obtained by extraction with water, a C₁-C₄ alcohol, hexane, or a mixed solvent thereof.

4. The method according to claim 1, wherein the pharmaceutical composition further comprises a walnut extract.

5. The method according to claim 1, wherein the extract comprises oleic acid, linoleic acid, and linolenic acid in a weight ratio of 1-2.5:4-8:1.

6. The method according to claim 5, wherein the extract comprises oleic acid, linoleic acid, and linolenic acid in a weight ratio of 1.25-1.35:4.5-6.8:1.

7. The method according to claim 1, wherein the compressed extract, the solvent extract, or the fraction of the solvent extract of Juglans mandshurica fruit comprises 1.98 to 2.98 wt % of palmitic acid, 0.53 to 0.79 wt % of stearic acid, 9.08 to 13.62 wt % of oleic acid, 44.83 to 67.25 wt % of linoleic acid, and 6.93 to 10.39 wt % of linolenic acid.

8. The method according to claim 1, wherein the stroke is an ischemic stroke.

9. A method of food composition for preventing or ameliorating stroke in a subject in need thereof, comprising administering to the subject an effective amount of a food composition comprising a compressed extract, a solvent extract, or a fraction of the solvent extract of Juglans mandshurica fruit as an active ingredient.

10. The method according to claim 9, wherein the food composition is a functional health food.

11. A method of treating stroke in a subject in need thereof, comprising administering a compressed extract, a solvent extract, or a fraction of the solvent extract of Juglans mandshurica fruit as an active ingredient to the subject.

12. (canceled)
